(19) United States
(12) Patent Application Publication
(10) Pub. No.: US 2009/0131897 A1
(43) Pub. Date: May 21, 2009

(54) Releasable Mechanical Fastener Device and Use Thereof

(75) Inventors: Fabrizio LORI, Cereste di Virgilio (IT); Graziano BORTOLETTO, Zero Branco (IT)

Correspondence Address: SALTAMAR INNOVATIONS, 30 FERN LANE, SOUTH PORTLAND, ME, 04106, US

(73) Assignee: NUOVA PANSAC S.P.A., Milano (IT)

(21) Appl. No.: 12/339,540

(22) Filed: Dec. 19, 2008

Related U.S. Application Data
(63) Continuation-in-part of application No. PCT/EP2007/005822, filed on Dec. 19, 2008.

(30) Foreign Application Priority Data
Jul. 15, 2006 (IT) MI 2006 A 001300

(51) Int. Cl. A61F 13/15

(52) U.S. Cl. 604/367; 604/385.23; 604/391

(57) ABSTRACT

A releasable mechanical fastener is provided using the hook or mushroom male strap, and instead of a dedicated female strap, utilizes a support and operational surface of the item to be fastened as the female material. A clothing or personal hygiene item is further provided, by way of example an infant diaper or an incontinence pad, utilizing a closable strap. The female material of the invention is not confined and not applied, but rather occupies and constitutes the support and operation surface of the device: it actually forms such surface of such clothing or personal hygiene item.

### RELATED APPLICATIONS

This application claims the right of priority from, and is a continuation in parts of, PCT patent application No. PCT/EP2007/005822, FILED Jul. 2, 2007, which in turn claims priority from Italian patent application No. MIMI 2006 A 001300, filed Jul. 5, 2006. Both of the above identified patent applications are incorporated herein by reference in their entirety.

### FIELD OF THE INVENTION

The invention relates in general to mechanical fasteners and more specifically to two element releasable mechanical fastener which can be pressed together for mutual engagement and released by a tearing action and the to several articles using such articles.

### BACKGROUND

The device disclosed below finds application, for instance, in the field of clothing and particularly of personal hygiene items, such as infant diapers and incontinence pads.

Fastener devices are known in the art—available with trade names such as Velcro® and Microplast®—which comprise a male member, with generally and/or substantially hook- or mushroom-shaped elements thereon, and a female member, with generally and/or substantially loop-shaped elements thereon, for mutual engagement.

Female straps are known to be formed, particularly, by attaching nonwoven fabrics having particular fiber arrangements to facilitate engagement to a support film.

Nevertheless, the use of female straps as a means for engagement of the hook elements of the male straps has certain drawbacks.

Prior art clothing and personal hygiene items, such as diapers and incontinence pads, require the use of a given amount of female material, placed on a support of adequate size in a predetermined position of the clothing or personal hygiene item, whose positioning occurs in a specific manufacturing step.

These aspects have a considerable effect on the manufacture of the final product and the associated costs.

### SUMMARY

Our system overcomes the above problematic aspects and provides flexibility in terms of both operation and use, in addition to achieving considerable cost reduction.

The female material of the invention is not confined and not applied, but rather occupies and constitutes the support and operation surface of the device: it actually forms such surface.

A novel fastener system will thus be described hereinunder in greater detail, which requires no reinforcement parts to preserve the integrity of the product over the mechanical fastening area.

Furthermore, the direct engagement of the male material as described above with the female material of the invention provides considerable resistances to tearing as well as enabling opening and closing the fastener device several times, without reducing its holding effect, which remains thus unchanged.

Hence, the invention fulfils the object of obviating at least some of prior art problems and particularly the ones described above.

Further advantages may be obtained by the additional features described

### BRIEF DESCRIPTION OF THE DRAWINGS

The invention will be now described with reference to the attached drawings in which:

FIG. 1 is a cross sectional view of a female surface of a fastener device;

FIG. 2 is an enlarged detail showing a male strap engaged with a female strap;

FIG. 3 is a perspective view of a fastener device with a male strap-like member, in which the female member forms the support layer of the finished item.

FIG. 4 depicts a personal hygiene item such as diaper or an incontinence pad.

FIG. 5 depicts another view of a personal hygiene item such as a diaper or an incontinence pad.

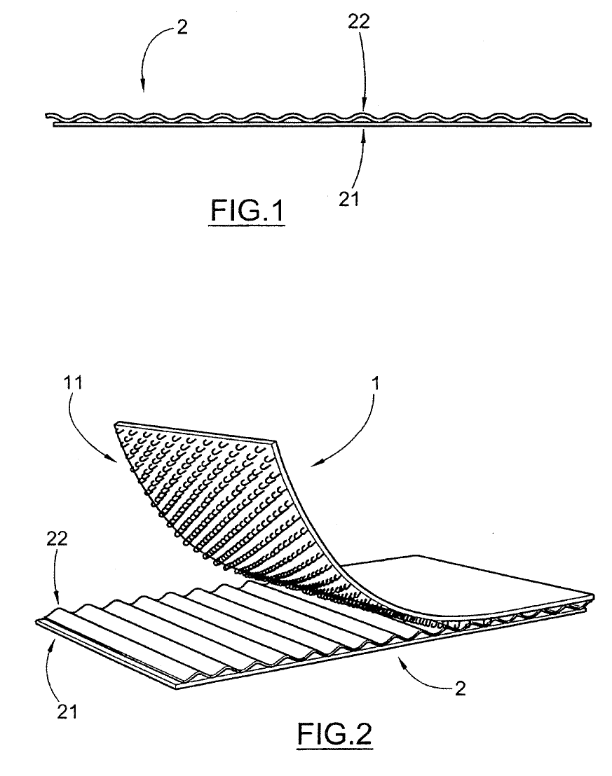

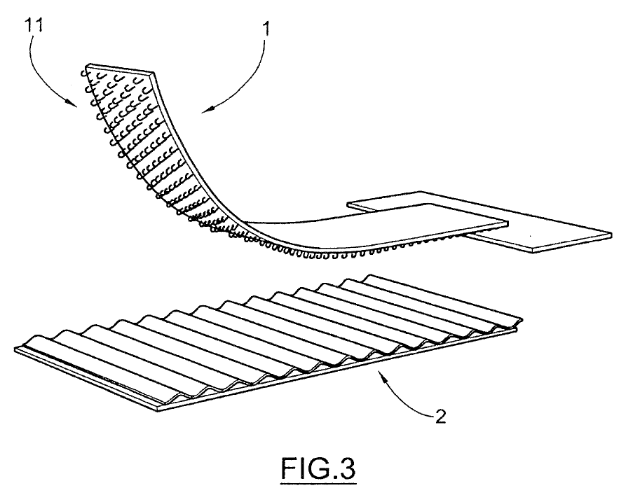

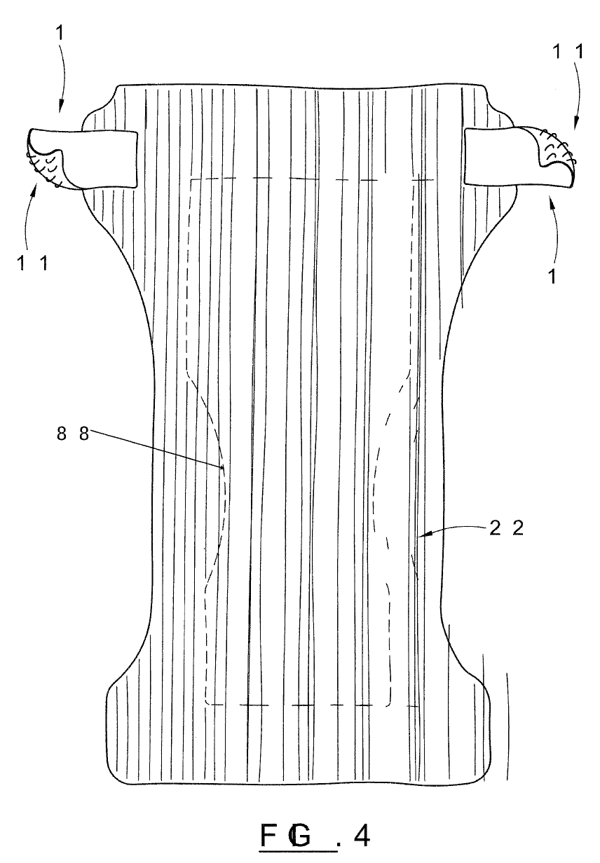

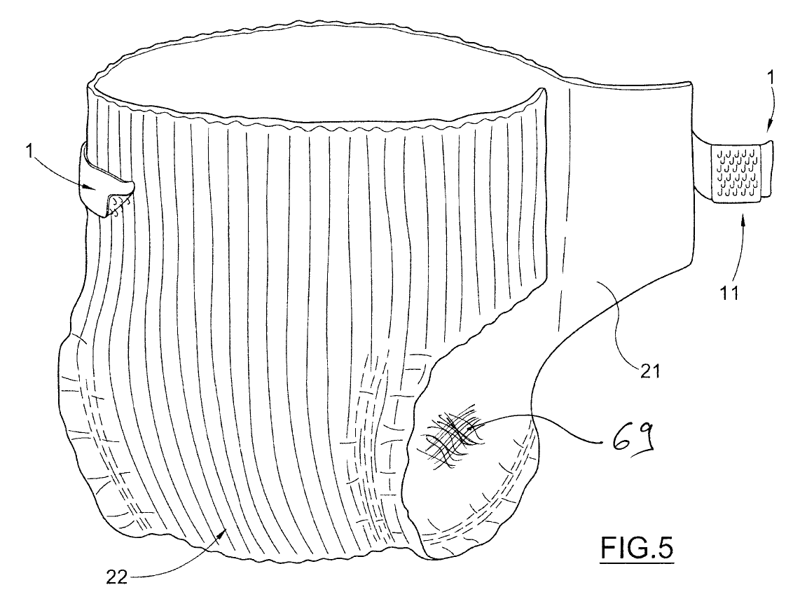

### DETAILED DESCRIPTION

Referring to the annexed drawings, the releasable mechanical fastener device comprises a first male strap having a plurality of hook elements 11 which project out of at least one side 12 of the first strap 1.

The hook elements 11 may have conventional shapes ad sizes.

A second member 2 (female strap) is further provided, which is a laminate material 21, 22, comprising a polymer-based film 21 and a nonwoven fabric layer 22, preferably spun-bonded, attached to the polymer film 21.

The nonwoven fabric 22 and the polymer-based film 21 may be joined together by using cool melt and/or hot melt adhesives, heat sealing, ultrasounds.

The manner for joining together the nonwoven fabric 22 and the film 21 are conventional and will not be further described.

The nonwoven fabric 22 of the second strap 2 has the peculiar characteristic of having a corrugated cross-sectional profile.

In the preferred embodiment, the corrugated profiles have a rectilinear extension, orthogonal to the longitudinal direction of the strap 1.

The corrugated cross-sectional profile may be obtained, for example, using a device as disclosed in U.S. Pat. No. 6,499,640 B2 by the applicant hereof.

Thus, the corrugated profile adds elasticity to the female member to make the product better adaptable and to further counteract the tangential force (especially in clothing items and diapers) that tends to peel the male strap off by assisting the engagement of the hooking and enhancing the closing effect.

Preferably, the corrugated profile has a tip-to-tip pitch from about 1.8 mm to about 3.5 mm and a depth from 1.7 mm to 4.5 mm.

Preferably, the total basis weight of the second “strap” 2 is of 26 g/m2 to 50 g/m2.

More preferably, the basis weight of the second “strap” 2 is of 35 g/m2 to 45 g/m2.

In a possible embodiment, this laminate material has a water-vapor permeability, based on D6701-01 standard, of at least 500 (g H₂O/m²)/24 h, and an air permeability, based on ATICELCA MC 18-74 and TAPPI T 460 om-88 standards, below 100 min/100 cc of air.

The film 21 (of the second strap 2) may have an olefinic base material with fillers—such as the one disclosed in EP-B1-1,226,013 by the applicant hereof—or an olefinic and elastomeric base material with fillers—such as the one disclosed in WO 2005/051635 by the applicant hereof.

This solution is particularly interesting because it imparts a high elasticity to the second member and reduces the risk that the laminate material may be torn.

The spun-bonded nonwoven fabric strap 22 may be formed of polypropylene produced by traditional catalysis or by metallocene catalysis.

For example, the nonwoven fabric may be made using, as a starting material, the polypropylene currently sold by Innovene as 100-ZA25 and 100-ZA35 or the polypropylene currently sold by BASELL as HP651S and HM562S and finally by BOREALIS as HF420FB, HG420FB, HG455FB, HH420FB and HH450FB, or polypropylenes having similar characteristics (polypropylene homopolymers having MFRs from 25 g/10′ to 35 g/10′ and Melting Points from 160° C. to 165° C.).

Preferably, the fibers of the nonwoven fabric have a fineness of 1.5 to 2.5 deniers.

The above laminate material 21, 22 (comprising a polymer-based film 21 and a corrugated nonwoven fabric layer 22) may be used directly as a material for forming clothing and personal hygiene items.

For instance, for infant diapers and incontinence pads, the laminate material 21, 22 may form the support layer for the absorbent material 88.

Optionally, a fibrous permeable sheet 69 (commonly known as topsheet) may be joined to the laminate material, in such a way to cover the absorbent material 88. The fibrous permeable sheet 69 may be made for example of a non woven material, and may be joined directly to the laminate material 21, 22 except where the absorbent material lies. For instance the fibrous permeable sheet can be joined to the laminate sheet 21, 22 by discontinuously spreading an adhesive on the nonwoven fabric. Preferably the permeable sheet has an average thickness of 0.01-0.02 mm.

In the preferred embodiments, only the male straps 1 need to be applied to the product, as the surface of the product acts as a female member adapted to act as a base for engagement for the hook or mushroom elements 11 of the male straps 1.

In this case, it is obviously preferable for the laminate material to have the above air permeability and water-vapor permeability properties.

In clothing items and personal hygiene items, the use of a support structure in which the polymer film 21 is formed of a mixture of elastomers and/or olefins is particularly advantageous as it provides improved adjustment of fastener devices.

1. A clothing or personal hygiene item, comprising:
a laminate comprising a polymer-based film and nonwoven fabric layer formed in a corrugated profile, said laminate forming at least one support and operation surface of said item;
an absorbent material being supported by said laminate;
at least one strap, having a plurality of hook or mushroom elements projecting therefrom, for engagement with said corrugations of said nonwoven fabric layer, wherein said engagement provides fastening action between said strap and said support and operation surface.

2. A clothing or personal hygiene item as claimed in claim 1, wherein said corrugated profile of said nonwoven fabric has a tip-to-tip pitch from 1.8 mm to 3.5 mm.

3. A clothing or personal hygiene item as claimed in claim 1, wherein said corrugated profile has a depth from 1.7 mm to 4.5 mm.

4. A clothing or personal hygiene item as claimed in claim 1 said nonwoven fabric comprises of fibers having a fineness from 1.5 deniers to 2.5 deniers.

5. A clothing or personal hygiene item as claimed in claim 1, wherein said laminate material has a water-vapor permeability, based on ASTM D6701-01 standard, of at least 500 (g H2O/m2)/24 h, and an air permeability, based on ATICELCA MC 18-74 and TAPPI T 460 om-88 standards, below 100 min/100 cc of air.

6. A clothing or personal hygiene item as claimed in claim 1, wherein said laminate material has a total basis weight of 26 g/m2 to 50 g/m2.

7. A clothing or personal hygiene item as claimed in claim 1, wherein said polymer-based film is an olefin- and elastomer-based material with fillers.

8. A clothing or personal hygiene item as claimed in claim 1, wherein said non-woven material is spun-bonded.

9. A personal hygiene item as claimed in claim 1, wherein said item is a diaper.

10. A personal hygiene item as claimed in claim 1, wherein said item is an incontinence pad.

11. A clothing or personal hygiene item as claimed in claim 1, wherein said polymer-based film is an olefin-based material with fillers.

12. A clothing or personal hygiene item as claimed in claim 1, wherein said polymer-based film is an elastomer-based material with fillers.

13. A clothing or personal hygiene item as claimed in claim 1, further comprising a liquid permeable fibrous sheet, said liquid permeable sheet lying proximal to said absorbent material and wherein said liquid permeable sheet and said laminate are joined together.

14. A clothing or personal hygiene item as claimed in claim 12 wherein said liquid permeable sheet and said absorbent material are non-congruent.
